(19) United States
(12) Patent Application Publication
(10) Pub. No.: US 2011/0257233 A1
(43) Pub. Date: Oct. 20, 2011

(54) BENZOISOTHIAZOLONES AS INHIBITORS OF PHOSPHOMANNOSE ISOMERASE

(75) Inventors: Nicholas D. P. Cosford, San Diego, CA (US); Hudson H. Freeze, San Diego, CA (US); Russell Dahl, Carlsbad, CA (US); Yalda Bravo, San Diego, CA (US); Vandana Sharma, San Diego, CA (US); Mie Ichikawa, San Diego, CA (US)

(73) Assignee: Sanford-Burnham Medical Research Institute, La Jolla, CA (US)

(21) Appl. No.: 13/051,910

(22) Filed: Mar. 18, 2011

Related U.S. Application Data
(60) Provisional application No. 61/315,854, filed on Mar. 19, 2010.
(60) Provisional application No. 61/315,789, filed on Mar. 19, 2010.

(51) Int. Cl. A61K 31/428; A61P 31/04; A61P 31/00; A61P 3/10; A61P 25/00; A61P 1/16; A61P 13/12; C07D 275/04; A61P 27/10

(52) U.S. Cl. 514/373; 548/209

(57) ABSTRACT

The disclosure provides new compounds and compositions thereof, and methods for treating or ameliorating a disorder relating to CDG-Ia. In particular, the disclosure provides benzoisothiazolone inhibitors of PMI, which have been synthesized and their ability to drive glycosylation has been demonstrated. The disclosure provides two synthetic routes for these compounds, including a new copper-catalyzed N-arylation reaction amenable to parallel derivitization. The disclosed compounds represent potent inhibitors of PMI, and their dose-dependent efficacy in cell-based models of glycosylation have been demonstrated. In addition, the disclosed compounds are selective over PMM and therefore, are useful in treating or ameliorating a disorder relating to CDG-Ia.

### CROSS REFERENCE TO RELATED APPLICATIONS

This application claims priority to U.S. Provisional Application No. 61/315,854, filed Mar. 19, 2010; and to U.S. Provisional Application No. 61/315,789, filed Mar. 19, 2010, the disclosure of each is hereby incorporated by reference in their entirety for all purposes.

### STATEMENT OF GOVERNMENT SUPPORT

This invention was made in part with government support under Grant Nos. U54 HG003916, R01DK55615 and R21HD062914 awarded by the National Institutes of Health (NIH). The government has certain rights in the invention.

### BACKGROUND OF THE INVENTION

1. Field of the Invention

The disclosure relates generally to benzoisothiazolone compounds and compositions thereof, and methods of using these compounds and compositions as inhibitors of phosphomannose isomerase (PMI).

2. Background Information

Glycosylation is the enzymatic process that attaches polysaccharides and oligosaccharide (glycans) to proteins and lipids. Glycosylation is a form of co-translational and post-translational modification, which produces the fundamental biopolymers found in cells (DNA, RNA, and proteins). Glycans serve a variety of structural and functional roles in membrane and secreted proteins. The majority of proteins synthesized in the rough endoplasmic reticulum (ER) undergo glycosylation. The carbohydrate chains attached to the target proteins serve various functions. For instance, some proteins do not fold correctly unless they are glycosylated first. Also, polysaccharides linked at the amide nitrogen on asparagine in a protein confer stability on some secreted glycoproteins. Experiments have shown that glycosylation in this case is not a strict requirement for proper folding, but the unglycosylated protein degrades more quickly. Glycosylation may also play a role in cell-cell adhesion (a mechanism employed by cells of the immune system).

A congenital disorder of glycosylation (previously called carbohydrate-deficient glycoprotein syndrome) is one of several rare inborn errors of metabolism where glycosylation of a variety of tissue proteins and/or lipids is deficient or defective. Congenital disorders of glycosylation are sometimes known as CDG syndromes. They often cause serious, sometimes fatal, malfunction of several different organ systems (especially the nervous system, muscles, and intestines) in affected infants. CDG can be classified as Types I and II (CDG-I and CDG-II), depending on the nature and location of the biochemical defect in the metabolic pathway relative to the action of oligosaccharyltransferase. Currently, seventeen CDG type-I variants have been identified and twelve variants of CDG Type-II have been described. The most common subtype is CDG-Ia (also referred to as PMM2-CDG), wherein a genetic defect leads to the loss or reduction of phosphomannomutase 2 (PMM) activity, the enzyme responsible for the conversion of mannose-6-phosphate (Man-6-P) into mannose-1-phosphate (Man-1-P), leading to defective N-glycosylation. The specific problems produced differ according to the particular abnormal synthesis involved. Common manifestations include ataxia, seizures, retinopathy, liver fibrosis, coagulapathies, failure to thrive, dysmorphic features, e.g. inverted nipples and subcutaneous fat pads, and strabismus. Often, cerebellar atrophy and hypoplasia are found in a MRI. Ocular abnormalities of CDG-Ia include myopia, infantile esotropia, delayed visual maturation, low vision, optic pallor, and reduced rod function on electrotino-graphy. In addition, CDG-1a, 1b, and Id cause congenital hyperinsulinism with hyperinsulinemic hypoglycemis in infancy. Currently, there is no therapy for CDG-Ia patients and the prognosis is extremely poor. The disclosure addresses these issues and further provides related advantages.

### SUMMARY OF THE INVENTION

The disclosure provides compounds and compositions thereof, and methods for treating or ameliorating a disorder relating to CDG-Ia. In particular, the disclosure provides benzoisothiazolone inhibitors of PMI, which have been synthesized and their ability to drive glycosylation has been demonstrated. The disclosure provides two synthetic routes for these compounds, including a new copper-catalyzed N-arylation reaction amenable to parallel derivitization. The disclosed compounds represent the most potent inhibitors of PMI to date, and their dose-dependent efficacy in cell-based models of glycosylation have been demonstrated. In addition, the disclosed compounds are selective over PMM and therefore, are useful in treating or ameliorating a disorder relating to CDG-Ia.

Thus, in one embodiment the disclosure provides a compound of Formula I:

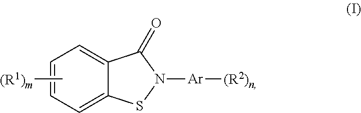

or a pharmaceutically acceptable salt or solvate thereof, wherein:

Ar is phenyl or naphthyl;

each R¹ is independently selected from hydrogen, amino, cyano, halogen, hydroxy, nitro, alkyl, alkenyl, alkynyl, trifluoroalkyl, cycloalkyl, and alkoxy;

each R² is independently selected from hydrogen, amino, cyano, halogen, hydroxy, nitro, alkyl, alkenyl, alkynyl, trifluoroalkyl, cycloalkyl, alkoxy, (CH₂)ⱼOR³, (CH₂)ⱼC(O)R³, (CH₂)ⱼC(O)OR³; (CH₂)jNR³R⁴ and (CH₂)ⱼC(O)NR³R⁴;

R³ and R⁴ are each independently selected from hydrogen and alkyl;

j is independently an integer selected from 0, 1, 2, 3, 4, 5, and 5; and

m and n are each independently an integer from 0, 1, 2, and 3.

In another embodiment, the disclosure provides methods for modulating the activity of phosphomannomutase 2 (PMM) and phosphomannose isomerase (PMI) by administering to a subject in need thereof, a therapeutically effective amount of the compound of Formula I.

In another embodiment, the disclosure provides methods for modulating the activity of phosphomannomutase 2 (PMM) by administering to a subject in need thereof, a therapeutically effective amount of the compound of Formula I.

In another embodiment, the disclosure provides methods for modulating the activity of phosphomannose isomerase (PMI) by administering to a subject in need thereof, a therapeutically effective amount of the compound of Formula I.

In another embodiment, the disclosure provides methods for inhibiting the activity of phosphomannose isomerase (PMI) by administering to a subject in need thereof, a therapeutically effective amount of the compound of Formula I.

In another embodiment, the disclosure provides methods for treating Congenital Disorder of Glycosylation Type Ia (CDG-Ia) by administering to a subject in need thereof, a therapeutically effective amount of the compound of Formula I.

In another embodiment, the disclosure provides methods for treating an microbial infection, by administering to a subject in need thereof, a therapeutically effective amount of the compound of Formula I or a pharmaceutical composition thereof.

In another embodiment, the disclosure provides methods for treating an microbial infection, by administering to a subject in need thereof, a therapeutically effective amount of the compound of Formula I or a pharmaceutical composition thereof, wherein the microbial infection is a bacterial infection.

In another embodiment, the disclosure provides methods for treating an microbial infection, by administering to a subject in need thereof, a therapeutically effective amount of the compound of Formula I or a pharmaceutical composition thereof, wherein the microbial infection is a bacterial infection, wherein the bacterial infection is a Gram negative bacterial infection.

In another embodiment, the disclosure provides methods for treating an microbial infection, by administering to a subject in need thereof, a therapeutically effective amount of the compound of Formula I or a pharmaceutical composition thereof, wherein the microbial infection is a bacterial infection, wherein the bacterial infection is a Gram negative bacterial infection, wherein the Gram negative bacterial infection is Pseudomonas aeruginosa infection.

In another embodiment, the disclosure provides methods for treating an microbial infection, by administering to a subject in need thereof, a therapeutically effective amount of the compound of Formula I or a pharmaceutical composition thereof, wherein the microbial infection is a fungal infection.

In another embodiment, the disclosure provides methods for treating an microbial infection, by administering to a subject in need thereof, a therapeutically effective amount of the compound of Formula I or a pharmaceutical composition thereof, wherein the microbial infection is a fungal infection, wherein the fungal infection is a Candida albicans or Cryptococcus neoformans infection.

In another embodiment, the disclosure provides methods for killing bacteria or fungi, wherein the bacteria or fungi are selected from gram-negative bacteria, gram-positive bacteria and yeast, the method comprising the step of administering to a subject in need thereof, a therapeutically effective amount of the compound of Formula I or a pharmaceutical composition thereof, wherein the contacting is for a time and under conditions effective to kill bacteria or fungi.

In another embodiment, the disclosure provides methods for killing bacteria or fungi, wherein the bacteria or fungi are selected from gram-negative bacteria, gram-positive bacteria and yeast, the method comprising the step of administering to a subject in need thereof, a therapeutically effective amount of the compound of Formula I or a pharmaceutical composition thereof, wherein the contacting is for a time and under conditions effective to kill bacteria or fungi, wherein the bacteria are Gram-negative bacteria.

In another embodiment, the disclosure provides methods for killing bacteria or fungi, wherein the bacteria or fungi are selected from gram-negative bacteria, gram-positive bacteria and yeast, the method comprising the step of administering to a subject in need thereof, a therapeutically effective amount of the compound of Formula I or a pharmaceutical composition thereof, wherein the contacting is for a time and under conditions effective to kill bacteria or fungi, wherein the bacteria are Gram-negative bacteria, wherein the Gram-negative bacteria are selected from Pseudomonas aeruginosa and Escherichia coli.

In another embodiment, the disclosure provides methods for killing bacteria or fungi, wherein the bacteria or fungi are selected from gram-negative bacteria, gram-positive bacteria and yeast, the method comprising the step of administering to a subject in need thereof, a therapeutically effective amount of the compound of Formula I or a pharmaceutical composition thereof, wherein the contacting is for a time and under conditions effective to kill bacteria or fungi, wherein the bacteria are Gram-positive bacteria.

In another embodiment, the disclosure provides methods for killing bacteria or fungi, wherein the bacteria or fungi are selected from gram-negative bacteria, gram-positive bacteria and yeast, the method comprising the step of administering to a subject in need thereof, a therapeutically effective amount of the compound of Formula I or a pharmaceutical composition thereof, wherein the contacting is for a time and under conditions effective to kill bacteria or fungi, wherein the bacteria are Gram-positive bacteria, wherein the Gram-positive bacteria are selected from Staphylococcus aureus and Streptococcus faecalis.

In another embodiment, the disclosure provides methods for killing bacteria or fungi, wherein the bacteria or fungi are selected from gram-negative bacteria, gram-positive bacteria and yeast, the method comprising the step of administering to a subject in need thereof, a therapeutically effective amount of the compound of Formula I or a pharmaceutical composition thereof, wherein the contacting is for a time and under conditions effective to kill bacteria or fungi, wherein the fungi are Candida albicans or Cryptococcus neoformans.

### DRAWINGS OF THE INVENTION

FIG. 1 illustrates phosphomannose isomerase (PMI) and phosphomannomutase (PMM) as important regulators of glycosylation. The benzothiazolone inhibitors were designed to inhibit PMI but not PMM, facilitating the accumulation of mannose-6-phosphate to drive glycosylation.

### DETAILED DESCRIPTION OF THE INVENTION

Abbreviations used herein have their conventional meaning within the chemical and biological arts. Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain, or cyclic hydrocarbon radical, or combinations thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, N-propyl, isopropyl, N-butyl, sec-butyl, tert-butyl, isobutyl, cyclobutyl, pentyl, cyclopentyl, hexyl, cyclohexyl, (cyclohexyl)methyl, cyclopropylmethyl, homologs and isomers of, for example, N-pentyl, N-hexyl, N-heptyl, N-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. Alkyl groups which are limited to hydrocarbon groups are termed “homoalkyl”.

The term “alkylene” by itself or as part of another substituent means a divalent radical derived from an alkyl, as exemplified, but not limited, by —CH₂CH₂CH₂CH₂—, —CH₂CH═CHCH₂—, —CH₂C═CCH₂—, —CH₂CH₂CH(CH₂CH₂CH₃)CH₂—. Typically, an alkyl (or alkylene) group will have from 1 to 24 carbon atoms. A “lower alkyl” or “lower alkylene” is a shorter chain alkyl or alkylene group, generally having eight or fewer carbon atoms.

As used herein, the terms “alkyl” and “alkylene” are interchangeable depending on the placement of the “alkyl” or “alkylene” group within the molecule.

The term “heteroalkyl,” by itself or in combination with another term, means, unless otherwise stated, a stable straight or branched chain, or cyclic hydrocarbon radical, or combinations thereof, consisting of at least one carbon atoms and at least one heteroatom selected from the group consisting of O, N, P, Si and S, and wherein the nitrogen, phosphorus, and sulfur atoms may optionally be oxidized and the nitrogen heteroatom may optionally be quaternized. The heteroatom(s) O, N, P and S and Si may be placed at any interior position of the heteroalkyl group or at the position at which alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃ and —CN. Up to two or three heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxo, alkylenedioxo, alkyleneamino, alkylenediamino, and the like). Still further, for alkylene and heteroalkylene linking groups, no orientation of the linking group is implied by the direction in which the formula of the linking group is written. For example, the formula —C(O)OR′— represents both —C(O)OR′— and —R′OC(O)—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. As used herein, the terms “heteroalkyl” and “heteroalkylene” are interchangeable depending on the placement of the “heteroalkyl” or “heteroalkylene” group within the molecule.

The terms “cycloalkyl” and “heterocycloalkyl”, by themselves or in combination with other terms, represent, unless otherwise stated, cyclic versions of “alkyl” and “heteroalkyl”, respectively. Additionally, for heterocycloalkyl, when the heteroatom is nitrogen, it can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. The terms “cycloalkylene” and “heterocycloalkylene” refer to the divalent derivatives of cycloalkyl and heterocycloalkyl, respectively. As used herein, the terms “cycloalkyl” and “cycloalkylene” are interchangeable depending on the placement of the “cycloalkyl” or “cycloalkylene” group within the molecule. As used herein, the terms “heterocycloalkyl” and “heterocycloalkylene” are interchangeable depending on the placement of the “heterocycloalkyl” or “heterocycloalkylene” group within the molecule.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “halo(Ci-C₄)alkyl” is mean to include, but not be limited to, trifluoromethyl, 2,2,2-trifluoroethyl, 4-chlorobutyl, 3-bromopropyl, and the like. As used herein, the terms “haloalkyl” and “haloalkylene” are interchangeable depending on the placement of the “haloalkyl” or “haloalkylene” group within the molecule.

The term “aryl” means, unless otherwise stated, a polyunsaturated, aromatic, hydrocarbon substituent which can be a single ring or multiple rings, which are fused together or linked covalently. The term “heteroaryl” refers to aryl groups (or rings) that contain from one to four heteroatoms (in each separate ring in the case of multiple rings) selected from N, O, and S, wherein the nitrogen and sulfur atoms are optionally oxidized, and the nitrogen atom(s) are optionally quaternized. For example, pyridine N-oxide moieties are included within the description of “heteroaryl.” A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, A-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, A-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent radicals of aryl and heteroaryl, respectively. As used herein, the terms “aryl” and “arylene” are interchangeable depending on the placement of the “aryl” and “arylene” group within the molecule. As used herein, the terms “heteroaryl” and “heteroarylene” are interchangeable depending on the placement of the “heteroaryl” and “heteroarylene” group within the molecule.

For brevity, the term “aryl” when used in combination with other terms (e.g., aryloxo, arylthioxo, arylalkyl) includes both aryl and heteroaryl rings as defined above. Thus, the term “arylalkyl” is meant to include those radicals in which an aryl group is attached to an alkyl group (e.g., benzyl, phenethyl, pyridylmethyl and the like) including those alkyl groups in which a carbon atom (e.g., a methylene group) has been replaced by, for example, an oxygen atom (e.g., phenoxymethyl, 2-pyridyloxymethyl, 3-(1-naphthyloxy)propyl, and the like). However, the term “haloaryl,” as used herein is meant to cover aryls substituted with one or more halogens.

Where a heteroalkyl, heterocycloalkyl, or heteroaryl includes a specific number of members (e.g., “3 to 7 membered”), the term “member” referrers to a carbon or heteroatom.

The term “oxo” as used herein means an oxygen that is double bonded to a carbon atom.

Each of the above terms (e.g., “alkyl,” “heteroalkyl,” “cycloalkyl, and “heterocycloalkyl”, “aryl,” “heteroaryl” as well as their divalent radical derivatives) are meant to include both substituted and unsubstituted forms of the indicated radical. Substituents for each type of radical are provided below.

Substituents for alkyl, heteroalkyl, cycloalkyl, heterocycloalkyl monovalent and divalent derivative radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to: —OR′, ═O, ═NR′, ═N—OR′, —NR′R″, —SR′, -halogen, —SiR′R″R′″, —OC(O)R′, —C(O)R′, —CO₂R′—C(O)NR″R′″, —OC(O)NR′R″, —NR′C(O)R″, —NR′—C(O)NR″R′″, —NR′C(O)OR″, —NR′—C(NR″R′″)═NR″″, —S(O)R′, —S(O)₂R′, —S(O)₂NR′R″, —NR′SO₂R″, —CN and —NO₂ in a number ranging from zero to (2m′+1), where m¹ is the total number of carbon atoms in such radical. R′, R″, R′″ and R″″ each independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound of the disclosure includes more than one R group, for example, each of the R groups is independently selected as are each R¹, R″, R′″ and R″″ groups when more than one of these groups is present. When R¹ and R″ are attached to the same nitrogen atom, they can be combined with the nitrogen atom to form a A-, 5-, 6-, or 7-membered ring. For example, —NR¹R″ is meant to include, but not be limited to, 1-pyrrolidinyl and A-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for alkyl radicals above, exemplary substituents for aryl and heteroaryl groups (as well as their divalent derivatives) are varied and are selected from, for example: halogen, —OR′, —NR′R″, —SR′, -halogen, —SiR′R″R′″, —OC(O)R′, —C(O)R′, —CO₂R′, —C(O)NR′R″, —OC(O)NR′R″, —NR′C(O)R″, —NR′—C(O)NR″R′″, —NR′C(O)OR″, —NRC(NR′R″R′″)═NR″″, —NRC(NR′R″)═NR′″, —S(O)R′, —S(O)₂R′, —S(O)₂NR′R″, —NR′SO₂R″, —CN and —NO₂, in a number ranging from zero to the total number of open valences on aromatic ring system; and where R′, R″, R′″ and R″″ are independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound of the disclosure includes more than one R group, for example, each of the R groups is independently selected as are each R′, R″, R′″ and R″″ groups when more than one of these groups is present.

Two of the substituents on adjacent atoms of aryl or heteroaryl ring may optionally form a ring of the formula -T-C(O)—(CRR′)_{q}—U—, wherein T and U are independently —NR—, —O—, —CRR′— or a single bond, and q is an integer of from 0 to 3. Alternatively, two of the substituents on adjacent atoms of aryl or heteroaryl ring may optionally be replaced with a substituent of the formula -A-(CH₂)ᵣ—B—, wherein A and B are independently —CR′R″—, —O—, —NR′—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituents on adjacent atoms of aryl or heteroaryl ring may optionally be replaced with a substituent of the formula —(CR′R″)ₛ—X′—(C″R′″)_{d}—, where s and d are independently integers of from O to 3, and X′ is —O—, —NR′—, —S—, —S(O)—, —S(O)₂—, or —S(O)₂NR′—. The substituents R′, R″, and R′″ are independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

An “aminoalkyl” as used herein refers to an amino group covalently bound to an alkylene linker. The amino group is —NR′R″, wherein R′ and R″ are typically selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

A “substituent group,” as used herein, means a group selected from at least the following moieties: (A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and (B) alkyl, heteroalkyl, cycloalkyl, heterocycloalkyl, aryl, and heteroaryl, substituted with at least one substituent selected from: (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and (ii) alkyl, heteroalkyl, cycloalkyl, heterocycloalkyl, aryl, and heteroaryl, substituted with at least one substituent selected from: (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and (b) alkyl, heteroalkyl, cycloalkyl, heterocycloalkyl, aryl, or heteroaryl, substituted with at least one substituent selected from oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “lower substituent group,” as used herein means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

The neutral forms of the compounds are regenerated by contacting the salt with a base or acid and isolating the parent compound in the conventional manner. The parent form of the compound differs from the various salt forms in certain physical properties, such as solubility in polar solvents.

Certain compounds of the disclosure can exist in unsolvated forms as well as solvated forms, including hydrated forms. In general, the solvated forms are equivalent to unsolvated forms and are encompassed within the scope of the disclosure. Certain compounds of the disclosure may exist in multiple crystalline or amorphous forms. In general, all physical forms are equivalent for the uses contemplated by the disclosure.

Certain compounds of the disclosure possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the disclosure. The compounds of the disclosure do not include those which are known in art to be too unstable to synthesize and/or isolate. The disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure. Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the disclosure.

The compounds of the disclosure may also contain unnatural proportions of atomic isotopes at one or more of atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the disclosure, whether radioactive or not, are encompassed within the scope of the disclosure.

The term “pharmaceutically acceptable salts” is meant to include salts of active compounds which are prepared with relatively nontoxic acids or bases, depending on the particular substituent moieties found on the compounds described herein. When compounds of the disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, mono-hydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science, 66:1-19 (1977)). Certain specific compounds of the disclosure contain both basic and acidic functionalities that allow the compounds to be converted into either base or acid addition salts.

In addition to salt forms, the disclosure provides compounds, which are in a prodrug form. Prodrugs of the compounds described herein are those compounds that readily undergo chemical changes under physiological conditions to provide the compounds of the disclosure. Additionally, prodrugs can be converted to the compounds of the disclosure by chemical or biochemical methods in an ex vivo environment. For example, prodrugs can be slowly converted to the compounds of the disclosure when placed in a transdermal patch reservoir with a suitable enzyme or chemical reagent.

The terms “a,” “an,” or “a(n)”, when used in reference to a group of substituents herein, mean at least one. For example, where a compound is substituted with “an” alkyl or aryl, the compound is optionally substituted with at least one alkyl and/or at least one aryl. Moreover, where a moiety is substituted with an R substituent, the group may be referred to as “R-substituted.” Where a moiety is R-substituted, the moiety is substituted with at least one R substituent and each R substituent is optionally different.

Description of compounds of the disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The terms “treating” or “treatment” in reference to a particular disease includes prevention of the disease.

The symbol >˜w- denotes the point of attachment of a moiety to the remainder of the molecule.

As used herein, a therapeutically effective amount of a disclosed compound means that amount which

As used herein, the term “subject” refers to an animal, for example, a mammal or a human, who has been the object of treatment, observation or experiment.

As used herein, the term “therapeutically effective amount” means that amount of active compound or pharmaceutical agent that elicits the biological or medicinal response in a tissue system, animal or human that is being sought by a researcher, veterinarian, medical doctor or other clinician, which includes alleviation, prevention, treatment, or the delay of the onset or progression of the symptoms of the disease or disorder being treated.

An effective amount of the disclosed compound can be administered in an amount of between about 0.01 to about 100 mg/kg body weight. In certain aspects, the disclosed compounds can be administered at a concentration of about 0.1 to about 50 mg/kg; in other aspects, the disclosed compounds can be administered at a concentration of about 0.1 to 25 mg/kg; in other aspects, the disclosed compounds can be administered at a concentration of about 0.2 to 20 mg/kg; in other aspects, the disclosed compounds can be administered at a concentration of about 0.3 to 15 mg/kg; in other aspects, the disclosed compounds can be administered at a concentration of about 0.4 to 10 mg/kg; in other aspects, the disclosed compounds can be administered at a concentration of about 0.5 to 5 mg/kg; in other aspects. It will be understood that the disclosure provides a basis for further studies in humans to more precisely determine effective amounts in humans. Doses used for rodent studies provide a basis for the ranges of doses indicated herein for humans and other mammals.

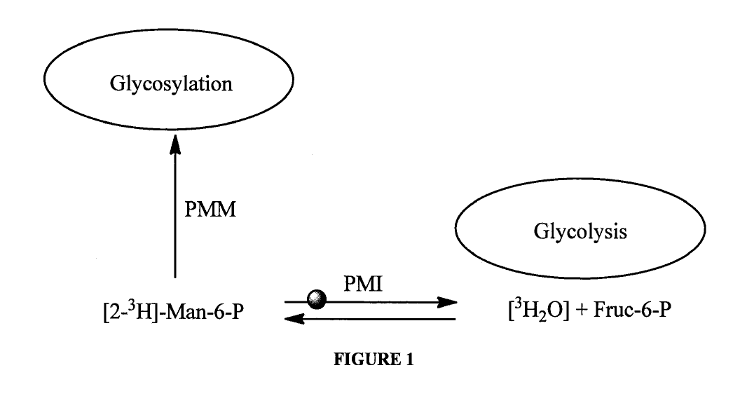

Congenital Disorder of Glycosylation Type la (CDG-Ia) is a rare autosomal recessive metabolic disorder with multisystemic symptoms where patients have decreased activity of phosphomannomutase 2 (PMM). This reduction in PMM activity impairs the conversion of mannose-6-phosphate (Man-6-P) to mannose-1-phosphate leading to defective N-glycosylation. It is hypothesized that that CDG-Ia patients may benefit from dietary mannose supplementation combined with inhibition of phosphomannose isomerase (PMI) using small molecule inhibitors selective for PMI over PMM, thus driving the metabolic flux into the glycosylation pathway (FIG. 1). To date, inhibitors of PMI are scarce, in which the only reported inhibitors are either substrate based or show weak inhibition. In addition, no cell-based efficacy or selectivity over PMM has been reported for any PMI inhibitors. However, it has been found that benzoisothiazolone derivatives of Formula I:

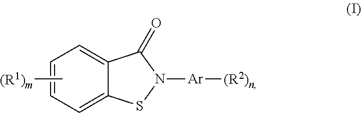

or a pharmaceutically acceptable salt or solvate thereof, wherein Ar is phenyl or naphthyl; and each of R¹, R², m, and n are as described herein, are PMI-selective inhibitors of human PMI and therefore, these compounds are useful for treating CDG-Ia.

Thus, in one embodiment the disclosure provides compounds having Formula I, or a pharmaceutically acceptable salt or solvate thereof, wherein:

Ar is phenyl or naphthyl;

each R¹ is independently selected from hydrogen, amino, cyano, halogen, hydroxy, nitro, alkyl, alkenyl, alkynyl, trifluoroalkyl, cycloalkyl, and alkoxy;

each R² is independently selected from hydrogen, amino, cyano, halogen, hydroxy, nitro, alkyl, alkenyl, alkynyl, trifluoroalkyl, cycloalkyl, alkoxy, (CH₂)ⱼOR³, (CH₂)ⱼC(O)R³, (CH₂)ⱼC(O)OR³; (CH₂)jNR³R⁴ and (CH₂)ᵢC(O)NR³R⁴;

R³ and R⁴ are each independently selected from hydrogen and alkyl;

j is independently an integer selected from 0, 1, 2, 3, 4, 5, and 5; and

m and n are each independently an integer from 0, 1, 2, and 3.

In another aspect the disclosure provides compounds of Formula I, wherein Ar is phenyl; each R1 is independently selected from hydrogen and halogen; and each R2 is independently selected from hydrogen, alkyl, trifluoroalkyl, halogen, OR3, C(O)R3, C(O)OR3; and NR3R4.

In another aspect the disclosure provides compounds of Formula I, wherein R1 is independently selected from hydrogen, fluoro, chloro, bromo, and iodo; and each R2 is independently selected from hydrogen, CH3, CF3, fluoro, chloro, bromo, iodo, OCH3, C(O)CH3, C(O)OCH3; and N(CH3)2.

In another aspect the disclosure provides compounds of Formula I, wherein the compound is:

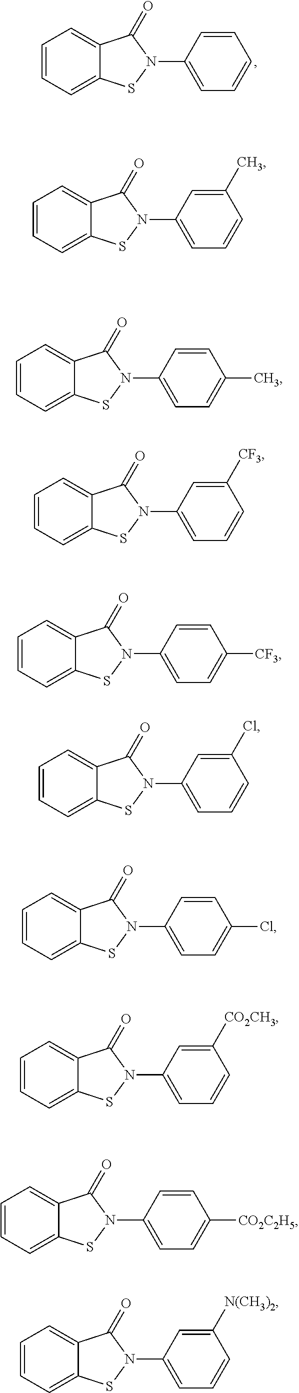

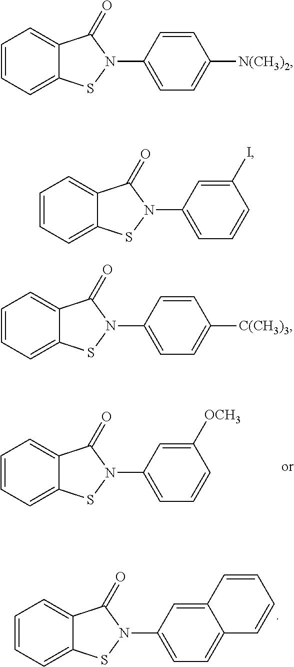

In another aspect the disclosure provides compounds of Formula I, wherein the compound is:

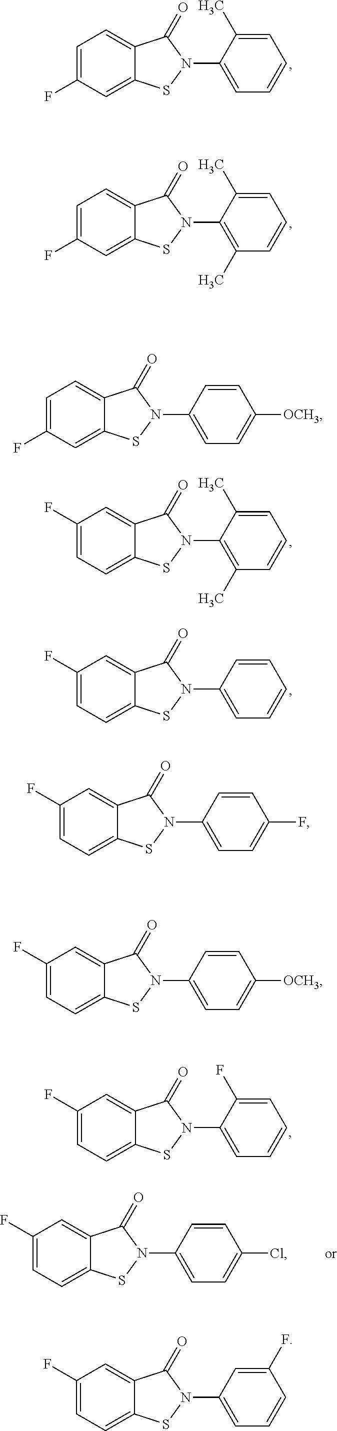

In another aspect the disclosure provides methods for modulating the activity of phosphomannomutase 2 (PMM) and phosphomannose isomerase (PMI) by administering to a subject in need thereof, a therapeutically effective amount of the compound of Formula I.

In another aspect the disclosure provides methods for modulating the activity of phospho-mannomutase 2 (PMM) by administering to a subject in need thereof, a therapeutically effective amount of the compound of Formula I.

In another aspect the disclosure provides methods for modulating the activity of phospho-mannose isomerase (PMI) by administering to a subject in need thereof, a therapeutically effective amount of the compound of Formula I.

In another aspect the disclosure provides methods for inhibiting the activity of phospho-mannose isomerase (PMI) by administering to a subject in need thereof, a therapeutically effective amount of the compound of Formula I.

In another aspect the disclosure provides methods for treating Congenital Disorder of Glycosylation Type Ia (CDG-Ia) by administering to a subject in need thereof, a therapeutically effective amount of the compound of Formula I.

In another aspect the disclosure provides methods for treating Congenital Disorder of Glycosylation Type Ia (CDG-Ia) by administering to a subject in need thereof, a therapeutically effective amount of the compound of Formula I, wherein the CDG-Ia includes ataxia, seizures, retinopathy, liver fibrosis, coagulapathies, failure to thrive, dysmorphic features, and/or strabismus.

In another aspect the disclosure provides methods for treating Congenital Disorder of Glycosylation Type Ia (CDG-Ia) by administering to a subject in need thereof, a therapeutically effective amount of the compound of Formula I, wherein the CDG-Ia includes myopia, infantile esotropia, delayed visual maturation, low vision, optic pallor, and/or reduced rod function on electrotinography.

In another aspect the disclosure provides methods for treating Congenital Disorder of Glycosylation Type Ia (CDG-Ia) by administering to a subject in need thereof, a therapeutically effective amount of the compound of Formula I, wherein the CDG-Ia is congenital hyperinsulinism with hyperinsulinemic hypoglycemis in infancy.

The mechanism of action of antimicrobials vary. However, they are generally believed to function in one or more of the following ways: by inhibiting cell wall synthesis or repair; by altering cell wall permeability; by inhibiting protein synthesis; or by inhibiting synthesis of nucleic acids. For example, beta-lactam antibacterials act through inhibiting the essential penicillin binding proteins (PBPs) in bacteria, which are responsible for cell wall synthesis. As another example, quinolones act, at least in part, by inhibiting synthesis of DNA, thus preventing the cell from replicating.

The pharmacological characteristics of antimicrobials, and their suitability for any given clinical use, vary. For example, the classes of antimicrobials (and members within a class) may vary in 1) their relative efficacy against different types of microorganisms, 2) their susceptibility to development of microbial resistance and 3) their pharmacological characteristics, such as their bioavailability, and biodistribution. Accordingly, selection of an appropriate antibacterial (or other antimicrobial) in a given clinical situation requires analysis of many factors, including the type of organism involved, the desired method of administration, the location of the infection to be treated and other considerations.

The disclosure also provides methods of treating or preventing an infectious disorder in a human or other animal subject, by administering a safe and effective amount of a compound of Formula I to a subject. As used herein, an “infectious disorder” is any disorder characterized by the presence of a microbial infection. The methods of the disclosure are for the treatment of bacterial or fungal infections. Such infectious disorders include (for example) central nervous system infections, external ear infections, infections of the middle ear (such as acute otitis media), infections of the cranial sinuses, eye infections, infections of the oral cavity (such as infections of the teeth, gums and mucosa), upper respiratory tract infections, lower respiratory tract infections, including pneumonia, genitourinary infections, gastrointestinal infections, gynecological infections, septicemia, sepsis, peritonitis, bone and joint infections, skin and skin structure infections, bacterial endocarditis, burns, antibacterial/antifungal prophylaxis of surgery, and antibacterial/antifungal prophylaxis in post-operative patients or in immunosuppressed patients (such as patients receiving cancer chemotherapy, organ transplant patients, or HIV infected patients).

The compounds and compositions of this invention can be administered topically or systemically. Systemic application includes any method of introducing the compounds into the tissues of the body, e.g., intrathecal, epidural, intramuscular, transdermal, intravenous, intraperitoneal, subcutaneous, sublingual, rectal, and oral administration. The specific dosage of antimicrobial to be administered, as well as the duration of treatment, are mutually dependent. The dosage and treatment regimen will also depend upon such factors as the specific compound used, the resistance pattern of the infecting organism to the compound used, the ability of the compound to reach minimum inhibitory concentrations at the site of the infection, the nature and extent of other infections (if any), the personal attributes of the subject (such as weight), compliance with the treatment regimen, the age and health status of the patient, and the presence and severity of any side effects of the treatment.

Typically, for a human adult (weighing approximately 70 kilograms), from about 75 mg, from about 200 mg, from about 500 mg to about 30,000 mg, from about 500 mg to about 10,000 mg, from about 500 mg to about 3,500 mg of compound is administered per day. Treatment regimens may extend from about 1 day, or from about 3 to about 56 days, or from 3 about 20 days, in duration. Prophylactic regimens (such as avoidance of opportunistic infections in immuno-compromised patients) may extend 6 months, or longer, according to good medical practice. A method of parenteral administration is through intravenous injection. As is known and practiced in the art, all formulations for parenteral administration must be sterile. For mammals, especially humans, (assuming an approximate body weight of 70 kilograms) individual doses of from about 100 mg, or from about 500 mg to about 7,000 mg, or from about 1,000 mg to about 3,500 mg, is acceptable.

In some cases, such as generalized, systemic infections or in immune-compromised patients, the invention may be dosed intravenously. The dosage form is generally isotonic and at physiological pH. The dosage amount will depend on the patient and severity of condition, as well as other commonly considered parameters. Determination of such doses is well within the scope of practice for the skilled practitioner using the guidance given in the specification. Another method of systemic administration is oral administration. Individual doses of from about 20 mg, from about 100 mg to about 2,500 mg, or to about 500 mg.

Topical administration can be used to deliver the compounds systemically, or to treat a local infection. The amounts of compounds to be topically administered depends upon such factors as skin sensitivity, type and location of the tissue to be treated, the composition and excipient (if any) to be administered, the particular compounds to be administered, as well as the particular disorder to be treated and the extent to which systemic (as distinguished from local) effects are desired.

Thus, in another embodiment, the disclosure provides methods for treating an microbial infection, by administering to a subject in need thereof, a therapeutically effective amount of the compound of Formula I or a pharmaceutical composition thereof.

In another embodiment, the disclosure provides methods for treating an microbial infection, by administering to a subject in need thereof, a therapeutically effective amount of the compound of Formula I or a pharmaceutical composition thereof, wherein the microbial infection is a bacterial infection.

In another embodiment, the disclosure provides methods for treating an microbial infection, by administering to a subject in need thereof, a therapeutically effective amount of the compound of Formula I or a pharmaceutical composition thereof, wherein the microbial infection is a bacterial infection, wherein the bacterial infection is a Gram negative bacterial infection.

In another embodiment, the disclosure provides methods for treating an microbial infection, by administering to a subject in need thereof, a therapeutically effective amount of the compound of Formula I or a pharmaceutical composition thereof, wherein the microbial infection is a bacterial infection, wherein the bacterial infection is a Gram negative bacterial infection, wherein the Gram negative bacterial infection is Pseudomonas aeruginosa infection.

In another embodiment, the disclosure provides methods for treating an microbial infection, by administering to a subject in need thereof, a therapeutically effective amount of the compound of Formula I or a pharmaceutical composition thereof, wherein the microbial infection is a fungal infection.

In another embodiment, the disclosure provides methods for treating an microbial infection, by administering to a subject in need thereof, a therapeutically effective amount of the compound of Formula I or a pharmaceutical composition thereof, wherein the microbial infection is a fungal infection, wherein the fungal infection is a Candida albicans or Cryptococcus neoformans infection.

In another embodiment, the disclosure provides methods for killing bacteria or fungi, wherein the bacteria or fungi are selected from gram-negative bacteria, gram-positive bacteria and yeast, the method comprising the step of administering to a subject in need thereof, a therapeutically effective amount of the compound of Formula I or a pharmaceutical composition thereof, wherein the contacting is for a time and under conditions effective to kill bacteria or fungi.

In another embodiment, the disclosure provides methods for killing bacteria or fungi, wherein the bacteria or fungi are selected from gram-negative bacteria, gram-positive bacteria and yeast, the method comprising the step of administering to a subject in need thereof, a therapeutically effective amount of the compound of Formula I or a pharmaceutical composition thereof, wherein the contacting is for a time and under conditions effective to kill bacteria or fungi, wherein the bacteria are Gram-negative bacteria.

In another embodiment, the disclosure provides methods for killing bacteria or fungi, wherein the bacteria or fungi are selected from gram-negative bacteria, gram-positive bacteria and yeast, the method comprising the step of administering to a subject in need thereof, a therapeutically effective amount of the compound of Formula I or a pharmaceutical composition thereof, wherein the contacting is for a time and under conditions effective to kill bacteria or fungi, wherein the bacteria are Gram-negative bacteria, wherein the Gram-negative bacteria are selected from Pseudomonas aeruginosa and Escherichia coli.

In another embodiment, the disclosure provides methods for killing bacteria or fungi, wherein the bacteria or fungi are selected from gram-negative bacteria, gram-positive bacteria and yeast, the method comprising the step of administering to a subject in need thereof, a therapeutically effective amount of the compound of Formula I or a pharmaceutical composition thereof, wherein the contacting is for a time and under conditions effective to kill bacteria or fungi, wherein the bacteria are Gram-positive bacteria.

In another embodiment, the disclosure provides methods for killing bacteria or fungi, wherein the bacteria or fungi are selected from gram-negative bacteria, gram-positive bacteria and yeast, the method comprising the step of administering to a subject in need thereof, a therapeutically effective amount of the compound of Formula I or a pharmaceutical composition thereof, wherein the contacting is for a time and under conditions effective to kill bacteria or fungi, wherein the bacteria are Gram-positive bacteria, wherein the Gram-positive bacteria are selected from Staphylococcus aureus and Streptococcus faecalis.

In another embodiment, the disclosure provides methods for killing bacteria or fungi, wherein the bacteria or fungi are selected from gram-negative bacteria, gram-positive bacteria and yeast, the method comprising the step of administering to a subject in need thereof, a therapeutically effective amount of the compound of Formula I or a pharmaceutical composition thereof, wherein the contacting is for a time and under conditions effective to kill bacteria or fungi, wherein the fungi are Candida albicans or Cryptococcus neoformans.

In another aspect the disclosure provides methods for modulating antimicrobial (bacterial, fungal) activity by administering to a subject in need thereof, a therapeutically effective amount of the compound of Formula I. The compounds of the present invention are potent antimicrobial agents or are of use as intermediates in the preparation of such agents.

High throughput screening (HTS) of the 196,000 compound library from the NIH MLSMR was conducted to identify small molecule inhibitors of PMI. Among the active hits was discovered the known compound ebselen (2-phenyl-1,2-benzisoselenazol-3(2H)-one). Ebselen is a mimic of glutathione peroxidase and is being investigated as a possible treatment for reperfusion injury, stroke and tinnitus. Ebselen is a potent scavenger of hydrogen peroxide as well as hydroperoxides including membrane bound phospholipid and cholesterylester hydroperoxides. Ebselen was found to be a potent PMI inhibitor with an IC₅₀ of 0.19 μM, however, this compounds did not show the desired selectivity as it was also a potent inhibitor of PMM with an IC₅₀ of 0.67 μM. In addition, ebselen has been reported to have multiple biological and molecular actions. Furthermore, selenium toxicity has been shown to be manifested acutely and chronically in several in vivo models. However, it was found that replacement of selenium with sulfur afforded a new PMI inhibitor, the des-seleno analogue 1 shown below, that was significantly less potent than ebselen (6.4 μM vs 0.19 μM), but unlike ebselen, it was surprisingly and unexpectedly completely devoid of PMM inhibition at concentrations up to 20

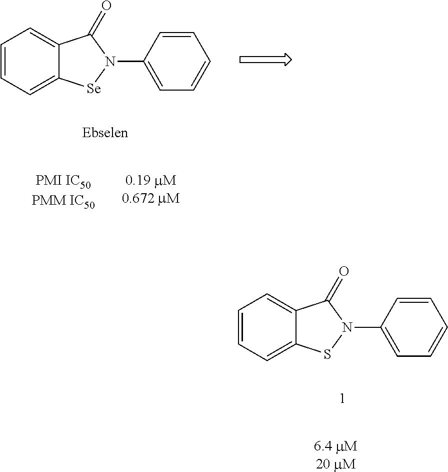

To facilitate SAR generation of the benzisothiazolone series, two chemical routes were developed to enable parallel synthesis of chemical libraries around this scaffold. The first route utilized chemistry developed by Conea, which involved a key cyclization step using phenyliodine bis(trifluoroacetate) (PIFA) to generate a N-acylnitrenium ion followed by intramolecular trapping by sulfur. This synthetic methodology allowed for substitution of both aromatic portions of the molecule and was utilized particularly to probe the effects of substitutions of the core benzisothiazolone ring (Scheme 1).

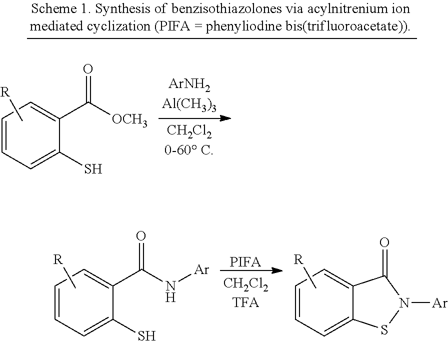

To efficiently assess the effects of substitutions of the pendant phenyl ring, a new copper-mediated Ullman-type N-arylation reaction was also developed. Beginning from commercially available benzoisothiazolone, this reaction involves the use of catalytic amounts of copper iodide and N,N′-dimethylethylenediamine as the ligand (Scheme 2).

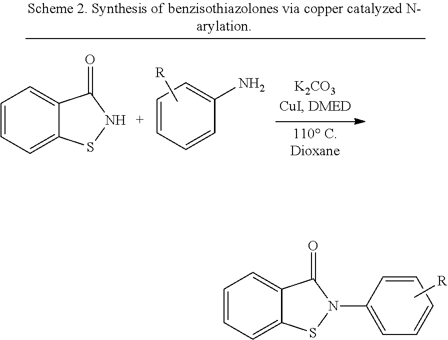

While the conditions shown above afforded the highest overall yields and substrate tolerances, it should be noted that other ligands and copper sources were screened. Generally, diamines and CuI gave the overall optimum yields, and CuCl and Cu₂O also yielded good results. This reaction was also preformed under microwave irradiation with acceptable product recovery in minutes. This is the first example of N-arylation chemistry of benzoisothiazolone and allowed rapid integration of the N-aryl species. Using a combination of both routes, the relevant sites to develop the structure-activity relationships (SAR) were investigated to afford optimal substitutions with respect to PMI potency, PMM selectivity, and cellular efficacy.

To determine the potential of the disclosed compounds to be effective in the accumulation of mannose-6-phosphate and thus improve glycosylation, several biochemical assays assessing PMI and PMM inhibition were used.

Table 1 shows the effects on PMI and PMM inhibition of substitutions on the pendant N-phenyl ring. Like the lead compound I, all of the synthesized derivatives showed selectivity for PMI over PMM. In general, para substitution was favored over meta, with a two- to three-fold increase in PMI inhibition seen. The exceptions to this trend included trifluoromethyl substitution (compounds 5 and 6) and ester substation (9 and 10). Some notable examples for overall potency and selectivity include compound 8 with a PMI IC₅₀ of 1.9 uM, and compound 12 which also showed comparable activity.

TABLE 1
SAR of N-phenyl ring substituents.*

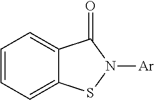

| Compound | Ar | PMI IC₅₀ (μM) | PMM IC₅₀ (μM) |
|---|---|---|---|
| 1 | Ph | 6.4 | >20 |
| 2 | 2-naphthyl | 9.4 | >20 |
| 3 | 3-CH₃—Ph | 6.0 | >20 |
| 4 | 4-CH₃—Ph | 3.6 | >20 |
| 5 | 3-CF₃—Ph | 3.4 | >20 |
| 6 | 4-CF₃—Ph | >20 | >20 |
| 7 | 3-Cl—Ph | 4.8 | >20 |
| 8 | 4-Cl—Ph | 1.9 | >20 |
| 9 | 3-CO₂CH₃—Ph | 4.9 | >20 |
| 10 | 4-CO₂C₂H₅—Ph | 7.2 | >20 |
| 11 | 3-(N(CH₃)₂—Ph | 8.5 | >20 |
| 12 | 4-(N(CH₃)₂—Ph | 1.9 | 13.3 |
| 13 | 3-I—Ph | 4.3 | >20 |
| 14 | 4-tert-Bu—Ph | 5.0 | >20 |
| 15 | 3-OCH₃—Ph | 3.7 | >20 |

*PMI and PMM assay data are the mean of at least three determinations.

Table 2 shows the effects of fluorine substitution at positions 5 and 6 on the core benzisothiazolone phenyl ring as assessed with respect to PMI potency and PMM selectivity. Generally, substitutions at these positions on this ring afforded an overall increase in PMI potency with all of the examples maintaining relative PMM selectivity. For example, compound 19, which displayed an unsubstituted phenyl ring and fluorine substitution at the 5 position of the fused aryl ring, had a PMI IC₅₀ of 1.3 μM as compared to the analogous des-fluoro derivative 1, which had a PMI IC₅₀ of 6.4 μM. In addition, both of these compounds were inactive against PMM when tested up to 20 μM. An exception to this trend was seen with the 4-chloro substituted compounds 24 and 8, which showed an IC₅₀ of 1.8 μM and 1.9 μM, respectively. It was in this series that the most potent compounds to date were observed, specifically the di-methyl substituted 17, with a fluorine in the 6-position; and the 4-methoxy derivative 22, with a fluorine in the 5 position. These derivatives showed PMI inhibition of 1.1 μM and 1.0 μM, respectively, representing a full fold better potency than the most potent derivatives from the previous des-methyl series. While inhibition of PMM was seen in these most potent examples, they still maintained a 7-9 fold selectivity for PMI.

TABLE 2
SAR of fluorine substituted benzoisothiazolones.*

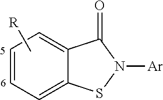

| Compound | R | Ar | PMI IC₅₀ (μM) | PMM IC₅₀ (μM) |
|---|---|---|---|---|
| 16 | 6-F | 2-CH₃—Ph | 2.9 | >20 |
| 17 | 6-F | 2,5-di-CH₃—Ph | 1.1 | 7.3 |
| 18 | 6-F | 4-OCH₃—Ph | 3.1 | 12.9 |
| 19 | 5-F | Ph | 1.3 | >20 |
| 20 | 5-F | 2,5-di-CH₃—Ph | 1.9 | >20 |
| 21 | 5-F | 4-F—Ph | 3.6 | >20 |
| 22 | 5-F | 4-OCH₃—Ph | 1.0 | 9.1 |
| 23 | 5-F | 2-F—Ph | 4.3 | >20 |
| 24 | 5-F | 4-Cl—Ph | 1.8 | >20 |
| 25 | 5-F | 3-F—Ph | 8.3 | >20 |

*PMI and PMM assay data are the mean of at least three determinations.

The importance of the heteroatoms in the benzisothiazolone ring was also investigated. To accomplish this, selected analogues were synthesized with removal or replacement of the nitrogen and sulfur atoms in the benzisothiazolone ring (compounds 26-28). All of these modifications abrogated all PMI activity when tested up to 10 μM in the enzyme assay, which supports the importance of these heteroatoms in this ring system (Scheme 2).

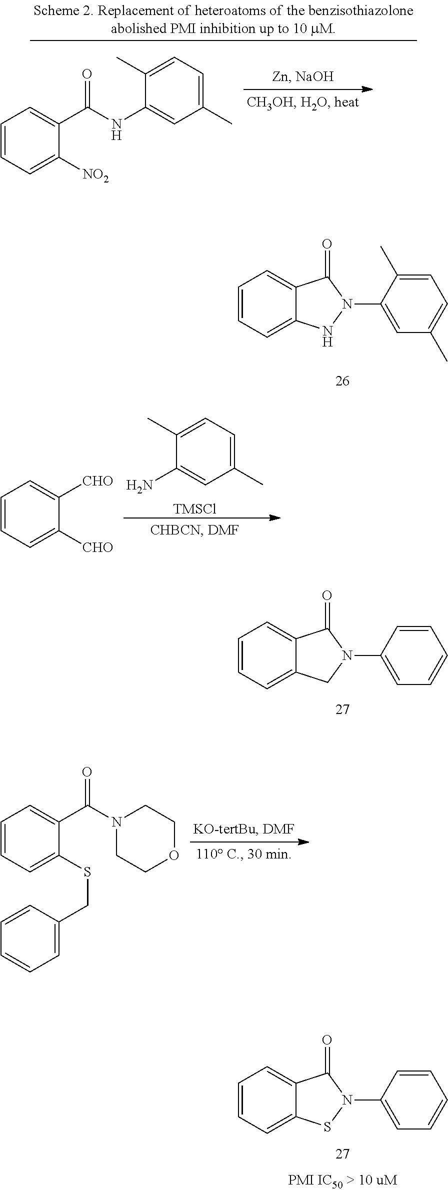

A cellular assay was developed and utilized to provide proof-of-concept for the potential of the optimized PMI inhibitors as CDG therapeutics. As shown in FIG. 1, tritiated mannose was used to directly measure the amount of mannose that is incorporated in proteins vs. mannose sent to glycolysis. As shown in Table 3, the inhibitors show a dose-dependent increase in protein glycosylation. Briefly. cells were preincubated with inhibitors and labeled with 3H-Mannose and 35S-Met/Cys. After washing and lysis, 3H- and 35S amounts were determined in the proteins.

TABLE 3
Cell based PMI Inhibitor data.*

| Compound | 12.5 | 25.0 μM | 50 μM |
|---|---|---|---|
| 20 | 1.9 | 2.3 | 3.2 |
| 23 | 9.1 | 0.7 | 2.5 |
| 21 | 2.8 | 2.9 | 2.5 |
| 18 | 1.5 | 2.5 | 32 |
| 16 | 2.7 | 2.8 | 1.7 |
| 8 | 11.6 | 17.3 | 6.1 |
| 12 | 5-F | 4-OMe—Ph | 1.0 |
| 25 | 9.4 | 12.5 | 16.8 |

*Shown is the fold increase in the amount of mannose incorporated in proteins vs glycolysis

ADME (absorption, distribution, metabolism, elimination) provides an efficient means of discovering potential issues with respect to bioavailability and the potential for in vivo efficiency. The microsomal stability assay measures a compound's potential to be metabolized by the liver and can also identify metabolic liabilities. The plasma stability assay gives essential information on whether a compound will be degraded in plasma. Finally, the Parallel Artificial Membrane Permeability Assay (PAMPA) measures compound diffusion rates through an artificial membrane to give valuable information on a compounds potential for intestinal absorption and tissue and cell permeability.

Selected PMI inhibitors were profiled in in vitro ADME assays to assess their drug-likeness and potential for systemic activity in animal models. Many of the benzisothiazolones were shown to have suitable properties for oral administration including acceptable metabolic and plasma stability, good permeability across artificial lipid membranes, and good solubility. The results of file specific ADME profiling assays are shown in Table 4.

TABLE 4
ADME profiles of selected PMI inhibitors.

| | | Plasma Stability^{b} | Microsomal Stability^{c} |
|---|---|---|---|
| | | human/mouse | human/mouse |
| | | (% remaining | (% remaining |
| Compound | Permeability^{a} | after 1 hour) | after 1 hour) |
| 20 | Medium | 96.7/99.4 | 100/85.04 |
| 23 | Medium | 35.7/106.7 | 27.66/98.40 |
| 21 | High | 0.0/71.4 | 24.93/81.24 |
| 16 | Medium | 49.7/117.5 | 90/90.03 |
| 22 | Low | 107.0/96.3 | 100/84.06 |

^{a}Permeabiliry is monitored by measuring the amount of compound that can diffuse through a lipid membrane.
^{b}Compounds are incubated with rat plasma and the amount of parent compound remaining is monitored by LCMS methods.
^{c}This assay is preformed by incubating test compounds with species-specific liver microsomes and monitoring degradation by LCMS.

The profiles of all of the synthesized analogues had acceptable and drug-like ADME profiles, showing acceptable aqueous solubility at physiological pH.

### EXAMPLES

The following examples are intended to illustrate but not limit the disclosed embodiments.

### Example 1

### Compound Collection Utilized in HTS

The compound library used in the high throughput screening assay (HTS) was supplied by the NIH Molecular Libraries Small Molecule Repository (MLSMR, http://www.mli.nih.gov/mlsnir). The MLSMR, funded by the NIH, is responsible for the selection of small molecules for HTS screening, their purchase and QC analysis, library maintenance and distribution within the NIH Molecular Libraries Screening Center Network (MLSCN. http://www.mli.nih.gov/mlscn). Both MLSMR and MLSCN are parts of the Molecular Libraries Initiatives (MLI, http://nihiroadmap.nih.gov/molecularlibraries) under the NIH Roadmap Initiative (www.nihroadmap.nih.gov). MLSMR compounds are acquired from commercial, and in part from academic and government sources and are selected based on the following criteria: samples are available for re-supply in 10 mg quantity, are at least 90% pure, have acceptable physicochemical properties and contain no functional groups or moieties which are known to generate artifacts in HTS (http://mlsnn.glpg.com). The compounds are selected to represent diversified chemical space with clusters of closely related analogs around them to aid in the HTS-based SAR analysis.

### Example 2

### High Throughput Screening Assays

For the HTS assay, 9 μl, of 2.2-fold PMI working solution was added to 384-well clear plates (Greiner 781101) containing 2 μL compound solutions; 2 μL of AF15394 solution in 10% DMSO and 10% DMSO alone were utilized for positive and negative control wells, respectively. The PMI working solution contained 50 mM Hepes, pH 7.4, 5 mM MgCl₂. 0.5 mM NADP+, 1 IU/mL PGI, 1.37/mL IU G6PDH and 0.9 μg/mL of PMI. After 60 min pre-incubation 9 μL of 2.2-fold mannose-6-phosphate working solution was added to the plates. Absorbance change was measured in a kinetic mode for 4 minutes at 340 nm. The slope of the progress curves was determined using linear regression. Compounds showing more than 50% inhibition were followed up with dose-response confirmation.

### Example 3

### Anti-Infective In Vitro Assays

The antimicrobial activity of compound 19 (5-Fluoro-2-phenylbenzo[d]isothiazol-3(2H)-one), was evaluated in the following anti-infective in vitro assays. The methods employed in this study were adapted from the scientific literature to maximize reliability and reproducibility. The reference standards were run as an integral part of each assay to ensure the validity of the results obtained. The assays were performed under conditions described below. The literature reference(s) for each assay are also provided as follows: Enza Di Modugno, Isabelle Erbetti, Livia Ferrari, Gianluca Galassi, Stephen M. Hammond, and Luigi xerri (1994) Microbiological properties of a new cephalosporin, BL-S 339: 7-(phenylacetyimidoyl-aminoacetamido)-3-(2-methyl-1,3,4-thiadiazol-5-ylthiomethyl)ceph-3-em-4-carboxylic acid. Antimicrobial Agents Chemotherapy 3: 40-48; Misiek, M., Pursiano, T. A., Leitner, F. and Price, K. E. (1973) In Vitro Activity of the Tribactam GV 104326 against Gram-Positive, Gram-Negative, and Anaerobic Bacteria. Antimicrobial Agents and Chemotherapy, 38: 2362-2368, 1994; Enza Di Modugno, Isabelle Erbetti, Livia Ferrari, Gianluca Galassi, Stephen M. Hammond, and Luigi xerri (1994) Microbiological properties of a new cephalosporin, BL-S 339: 7-(phenylacetyimidoyl-aminoacetamido)-3-(2-methyl-1,3,4-thiadiazol-5-ylthiomethyl)ceph-3-em-4-carboxylic acid. Antimicrobial Agents Chemotherapy 3: 40-48; and Misiek, M., Pursiano, T. A., Leitner, F. and Price, K. E. (1973) In vitro antibacterial activity of SM-7338, a carbapenem antibiotic with stability to dehydropeptidase I Antimicrobial Agents, Chemotherapy, 33 #2:215-222, 1989.

A summary of results meeting the significance criteria is as follows: The compound 19 was evaluated in the Candida albicans (ATCC 10231), Cryptococcus neoformans (ATCC 24067) and Pseudomonas aeruginosa (ATCC 27853) microbial assays at concentrations that range from 100 mg/ml to 0.03 mg/ml. Significant responses were noted in the Candida albicans and Cryptococcus neoformans microbial assay with a minimal inhibitory concentration of 3 mg/mL and 1 mg/mL respectively.

Summary of Significant Primary Results. Biochemical assay results are presented as the percent inhibition of specific binding or activity throughout the report. All other results are expressed in terms of that assay's quantitation method.

For primary assays, only the lowest concentration with a significant response judged by the assays' criteria, is shown in this summary.

Where applicable, either the secondary assay results with the lowest dose/concentration meeting the significance criteria or, if inactive, the highest dose/concentration that did not meet the significance criteria is shown.

Unless otherwise requested, primary screening in duplicate with quantitative data (e.g., IC50±SEM, Ki±SEM and nH) are shown where applicable for individual requested assays. In screening packages, primary screening in duplicate with semi-quantitative data (e.g., estimated IC50, Ki and nH) are shown where applicable (concentration range of 4 log units); available secondary functional assays are carried out (30 mM) and MEC or MIC determined only if active in primary assays >50% at 1 log unit below initial test concentration. Please see Experimental Results section for details of all responses.

Significant responses (50% inhibition or stimulation for Biochemical assays) were noted in the primary assays listed below:

PRIMARY TESTS

| | PRIMARY IN | | DOSE | | | QUANT |
|---|---|---|---|---|---|---|
| CAT. # | VITRO ASSAY | CLASS | (μg/mL) | CRITERIA | RESULTS | DATA |
| 640000 | Candida albicans | Fungi, | 3 | +/− | + | |
| | (ATCC 10231) | Mammalian | | | | |
| 647000 | Cryptococcus | Fungi, | 1 | +/− | + | |
| | neoformans | Mammalian | | | | |
| | (ATCC 24067) | | | | | |

EXPERIMENTAL RESULTS - FUNCTIONAL ASSAYS
MICROBIAL ASSAYS

| | | | | | CONC. | | |
|---|---|---|---|---|---|---|---|
| CAT. # | ASSAY NAME | CLASS | ROUTE | N = | (μg/mL) | CRITERIA | RESULT |
| 640000 | Candida albicans | Fungi | Vit | 2 | 100 | +/− | + |
| | (ATCC 10231) | | | | | | |
| | | | | | 30 | +/− | + |
| | | | | | 10 | +/− | + |
| | | | | | 3 | +/− | + |
| | | | | | 1 | +/− | − |
| | | | | | 0.3 | +/− | − |
| | | | | | 0.1 | +/− | − |
| | | | | | 0.03 | +/− | − |
| 647000 | Cryptococcus | Fungi | Vit | 2 | 100 | +/− | + |
| | neoformans | | | | | | |
| | (ATCC 24067) | | | | | | |
| | | | | | 30 | +/− | + |
| | | | | | 10 | +/− | + |
| | | | | | 3 | +/− | + |
| | | | | | 1 | +/− | + |
| | | | | | 0.3 | +/− | − |
| | | | | | 0.1 | +/− | − |
| | | | | | 0.03 | +/− | − |
| 614030 | Pseudomonas | Gram | Vit | 2 | 30 | +/− | − |
| | aeruginosa | Negative | | | | | |
| | (ATCC 27853) | | | | | | |
| | | | | | 10 | +/− | − |
| | | | | | 3 | +/− | − |
| | | | | | 1 | +/− | − |
| | | | | | 0.3 | +/− | − |
| | | | | | 0.1 | +/− | − |
| | | | | | 0.03 | +/− | − |
| | | | | | 30 | +/− | − |

Methods: Microbial In Vitro Assays

640000 Candida albicans (ATCC 10231); Culture Medium: Fluid Sabouraud Medium; Vehicle: 1% DMSO; Incubation Time/Temp: 20 hours @ 37° C.; Incubation Volume: 1 mL; Time of Assessment: 1 day; Quantitation Method: Turbidity Measurement.

614030 Pseudomonas aeruginosa (ATCC 27853): Culture Medium: Mueller-Hinton Broth; Vehicle: 1% DMSO; Incubation Time/Temp: 20 hours @37° C.; Incubation Volume: 1 mL; Time of Assessment: 1 day; Quantitation Method: Turbidity Measurement.

647000 Cryptococcus neoformans (ATCC 24067): Culture Medium: Yeast Mold Broth; Vehicle: 1% DMSO; Incubation Time/Temp: 2 days @37° C.; Incubation Volume: 1 mL; Time of Assessment: 2 days; Quantitation Method: Turbidity Measurement

Reference Compound Data—Microbial In Vitro Assays:

| CAT. | | | REFERENCE | CONCURRENT |
|---|---|---|---|---|
| # | ASSAY | CLASS | COMPOUND | (μg/mL) |
| 640000 | Candida | Fungi | Amphotericin | 0.1 |
| | albicans | | B Solubilized | |
| | (ATCC 10231) | | | |
| 647000 | Cryptococcus | Fungi | Amphotericin | 0.1 |
| | neoformans | | B Solubilized | |
| | (ATCC 24067) | | | |
| 614030 | Pseudomonas | Gram | Gentamicin | 0.3 |
| | aeruginosa | Negative | | |
| | (ATCC 27853) | | | |

### Example 4

General Synthetic Procedures. All solvents and chemicals used were purchased from Sigma-Aldrich, Acros, or Chembridge and were used as received without further purification. Purity and characterization of compounds were established by a combination of liquid chromatography-mass spectroscopy (LC-MS), and NMR analytical techniques and was >95% for all tested compounds. Silica gel column chromatography was carried out using prepacked silica cartridges from RediSep (ISCO Ltd.) and eluted using an Isco Companion system. ¹H NMR spectra were acquired on a Varian Inova 300 MHz. Chemical shifts are reported in ppm from residual solvent peaks (8 7.27 for CDCl₃ ¹H NMR). HPLC-MS analyses were performed on a Shimadzu 2010EV LCMS using the following conditions: Kromisil C:18 column (reverse phase 4.6 mm×50 mm): a linear gradient from 10% acetonitrile and 90% water to 95% acetonitrile and 5% water over 4.5 minutes; flow rate of 1 mL/minute; UV photo-diode array detection from 200 to 300 nm.

General Methods for the Synthesis of Benzoisothiazolone PMI Inhibitors.

General method A: To a stirred solution of the amine (900 mg. 5.95 mmol) in dichloromethane at 0° C. under nitrogen, Al(CH₃)₃ (6 mL, 2 M in THF) was added dropwise and the reaction was slowly warmed to room temperature. The mixture was stirred continuously for an additional 30 minutes. Methyl thiosalicylate (500 mg, 2.97 mmol) was added and the reaction was heated to 60° C. and refluxed overnight. The reaction was quenched with 5% aqueous HCl and dichloromethane was added (50 mL). The organic layer was separated and washed with saturated NaHCO₃ and brine and dried over Na₂SO₄. The solvents were removed by rotary evaporation and the products were isolated by flash chromatography or reverse phase HPLC and lyophilized to provide the final compounds, which were determined to be >95% pure by HPLC-UV, HPLC-MS, and ¹H NMR.

General method B: To a crimp top microwave vial was added the benzoisothiazolone (76 mg. 0.5 mmol), Aryl-X (1.05 mmol), K₂CO₃ (138 mg, 1.0 mmol), CuI (20 mol %), and DMEDA (20 mol %) in dioxane (5 mL). The reaction mixture was heated in the microwave at 195° C. for 7 minutes. Following filtration and evaporation of solvents, the products were isolated by flash chromatography or reverse phase HPLC and lyophilized to provide the final compounds, which were determined to be >95% pure by HPLC-UV, HPLC-MS, and ¹H NMR.

2-Phenyl-2-hydrobenzo[d]isothiazol-3-one (1). Prepared according to general method A (67%). ¹H NMR (300 MHz, CDCl₃): δ 7.32 (m, 1H), 7.51 (m, 3H), 7.57 (m, 1H), 7.67 (m, 3H), 8.09 (m, J=7.93, 1H).

2-(3-Methylphenyl)-2-hydrobenzo[d]isothiazol-3-one (3). Prepared according to general method B (22%). ¹H NMR (300 MHz, CDCl₃): δ 2.40 (s, 3H), 7.12 (d, 0.7=7.32, 1H), 7.33 (t, J=7.93, 1H), 7.49 (m, 4H), 7.62 (m, 1H), 8.08 (d, 0.7=7.93, 1H).

2-(4-Methylphenyl)-2-hydrobenzo[d]isothiazol-3-one (4). Prepared according to general method B (6%). ¹H NMR (300 MHz, CDCl₃): δ 2.37 (s, 3H). 7.26 (m, 3H), 7.42 (m, 1H), 7.55 (m, 2H), 7.64 (m, 1H), 8.08 (m, 1H).

2-[3(Trifluoromethyl)phenyl]-2-hydrobenzo[d]isothiazol-3-one (5). Prepared according to general method B (15%). ¹H NMR (300 MHz, CDCl₃): δ 7.46 (t, 0.7=7.9, 1H), 7.58 (m, 3H), 7.68 (m, 1H), 7.93 (d, J=7.9, 1H), 8.01 (s, 1H), 8.10 (d, J=7.9, 1H).

2-[4-(Trifluoromethyl)phenyl]-2-hydrobenzo[d]isothiazol-3-one (6). Prepared according to general method B (27%). ¹H NMR (300 MHz, CDCl₃): δ 7.59 (m, 5H), 7.90 (m. 2H). 8.10 (d, J=7.32, 1H).

2(3-Chlorophenyl)-2-hydrobenzo[d]isothiazol-3-one (7). Prepared according to general method B (15%). ¹H NMR (300 MHz, CDCl₃): δ 7.26 (m, 1H), 7.42 (m, 2H), 7.62 (m. 3H), 7.78 (m, 1H), 8.09 (m, 1H).

2-(4-Chlorophenyl)-2-hydrobenzo[d]isothiazol-3-one (8). Prepared according to general method B (12%). ¹H NMR (300 MHz, CDCl₃): δ 7.43 (m, 3H), 7.57 (d, J=7.93, 1H), 7.65 (m, 3H), 8.05 (d, J=7.9, 1H).

2-[4-(Dimethylamino)phenyl]-2-hydrobenzo[d]isothiazol-3-one (12). Prepared according to general method B (38%). ¹H NMR (300 MHz, CDCl₃): δ 2.97 (s, 6H), 6.75 (m, 2H), 7.42 (m, 3H), 7.54 (m, 1H), 7.60 (m, 1H), 8.07 (d, J=7.32, 1H).

2-(3-Iodophenyl)-2-hydrobenzo[d]isothiazol-3-one (13). Prepared according to general method B (18%). ¹H NMR (300 MHz, CDCl₃): δ 7.17 (t, J=7.93, 1H), 7.44 (m, 1H), 7.66 (m, 4H), 8.08 (m, 2H).

2[4-(tert-Butyl)phenyl]-2-hydrobenzo[d]isothiazol-3-one (14). Prepared according to general method B (42%). ¹H NMR (300 MHz, CDCl₃): δ 1.33 (s, 9H), 7.46 (m, 3H), 7.61 (m, 4H), 8.10 (d, J=7.93, 1H).

2-(3-Methoxyphenyl)-2-hydrobenzo[d]isothiazol-3-one (15). Prepared according to general method B (12%). ¹H NMR (300 MHz, CDCl₃): δ 3.84 (s, 3H), 6.81 (m, 1H), 7.25 (m, 1H), 7.36 (m, 2H), 7.43 (m, 1H), 7.57 (m, 1H), 7.65 (m, 1H), 8.09 (m, 1H).

6-Fluoro-2-o-tolylbenzo[d]isothiazol-3(2H)-one (16). Prepared according to general method A (55%). ¹H NMR (300 MHz, CDCl₃): δ 2.17 (s, 3H), 2.33 (s, 3H), 7.18 (m, 3H), 7.41 (m, 1H), 7.53 (m, 1H), 7.78 (dd, J=2.44, 7.93, 1H).

5-Fluoro-2-phenylbenzo[d]isothiazol-3(2H)-one (19). Prepared according to general method A (51%). ¹H NMR (300 MHz, CDCl₃): δ 7.33 (m, 1H), 7.45 (m, 3H), 7.54 (m, 1H), 7.67 (m, 2H), 7.77 (dd, J=7.93, 1H).

5-Fluoro-2-(4-fluorophenyl)-2-hydrobenzo[d]isothiazol-3-one (21). Prepared according to general method A (33%). ¹H NMR (300 MHz, CDCl₃): δ 7.15 (m, 2H), 7.42 (m, 1H), 7.54 (m, 1H), 7.62 (m, 2H), 7.76 (dd. J=2.44. 7.93, 1H).

5-Fluoro-2-(4-methoxyphenyl)-2-hydrobenzo[d]isothiazol-3-one (22). Prepared according to general method A (80%). ¹H NMR (300 MHz, CDCl₃): δ 3.83 (s, 3H), 6.97 (m 2H), 7.41 (m, 1H), 7.52 (m, 3H), 7.76 (dd. J=2.44. 7.93, 1H).

2-(4-Chlorophenyl)-5-fluoro-2-hydrobenzo[d]isothiazol-3-one (24). Prepared according to general method A (55%). ¹H NMR (300 MHz, CDCl₃): δ 7.42 (m, 3H), 7.54 (m, 1H), 7.63 (m, 2H), 7.76 (dd, 7=2.44, 7.93, 1H).

5-Fluoro-2-(3-fluorophenyl)-2-hydrobenzo[d]isothiazol-3-one (25). Prepared according to general method A (60%). ¹H NMR (300 MHz, CDCl₃): δ 7.02 (m, 1H), 7.43 (m, 3H), 7.54 (m, 2H), 7.75 (dd, J=2.44, 7.93, 1H).

2-Phenyl-1H-2-hydroindazol-3-one (26). A solution of o-nitrobenzaldehyde (242 mg, 1 mmol) in methanol (3 mL) was added to sodium hydroxide in water (4 mL) together with zinc dust. The resulting reaction mixture was then heated under reflux for 15 hours and filtered hot. The filtrate was concentrated to half and cooled. Any un-reacted material was removed by filtration. Filtrate was diluted with water and acidified with dilute HCl. The crude product precipitated and was collected by filtration and further purified by column chromatography using hexanes:ethyl acetate to afford 0.076 g (36%) of indazolone as a pale yellow solid. ¹H NMR (300 MHz, CDCl₃): δ 2.24 (s, 3H), 2.31 (s, 3H), 7.19 (m, 7H), 7.55 (m. 1H), 7.89 (m 1H).

2-(2,5-Dimethylphenyl)isoindolin-1-one (27). To a stirred solution of phthaladehyde (250 mg, 1.85 mmol) in CH₃CN:DMF the amine (230 μL, 1.85 mmol) was added followed by TMSCl (188 μL, 1.48 mmol). Stirred at room temperature overnight. (62%). ¹H NMR (300 MHz, CDCl₃): δ 2.18 (s, 3H), 2.31 (s, 3H), 4.70 (s, 2H), 7.18 (m, 2H), 7.52 (m, 3H), 7.93 (m, 1H).

Although the disclosure has been described with reference to the above examples, it will be understood that modifications and variations are encompassed within the spirit and scope of the disclosure.

What is claimed is:

1. A compound of Formula I: 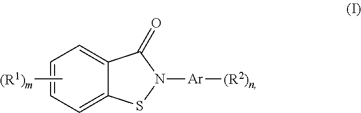
or a pharmaceutically acceptable salt or solvate thereof, wherein:
Ar is phenyl or naphthyl;
each R¹ is independently selected from hydrogen, amino, cyano, halogen, hydroxy, nitro, alkyl, alkenyl, alkynyl, trifluoroalkyl, cycloalkyl, and alkoxy;
each R² is independently selected from hydrogen, amino, cyano, halogen, hydroxy, nitro, alkyl, alkenyl, alkynyl, trifluoroalkyl, cycloalkyl, alkoxy, (CH₂)ⱼOR³, (CH₂)ⱼC(O)R³, (CH₂)ⱼC(O)OR³; (CH₂)jNR³R⁴ and (CH₂)ⱼC(O)NR³R⁴;
R³ and R⁴ are each independently selected from hydrogen and alkyl;
j is independently an integer selected from 0, 1, 2, 3, 4, 5, and 5; and
m and n are each independently an integer from 0, 1, 2, and 3.

2. The compound of claim 1, wherein Ar is phenyl; each R¹ is independently selected from hydrogen and halogen; and each R² is independently selected from hydrogen, alkyl, trifluoroalkyl, halogen, OR³, C(O)R³, C(O)OR³; and NR³R⁴.

3. The compound of claim 2, wherein R¹ is independently selected from hydrogen, fluoro, chloro, bromo, and iodo; and each R² is independently selected from hydrogen, CH₃, CF₃, fluoro, chloro, bromo, iodo, OCH₃, C(O)CH₃, C(O)OCH₃; and N(CH₃)₂.

4. The compound of claim 3, wherein the compound is:

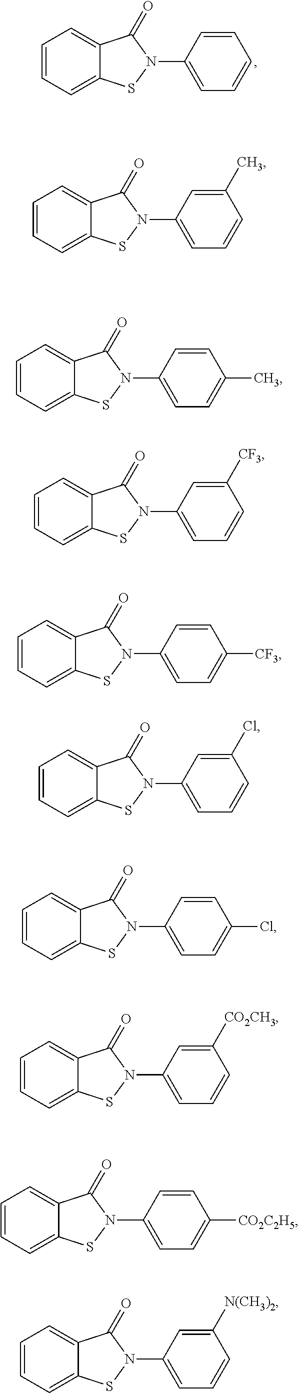

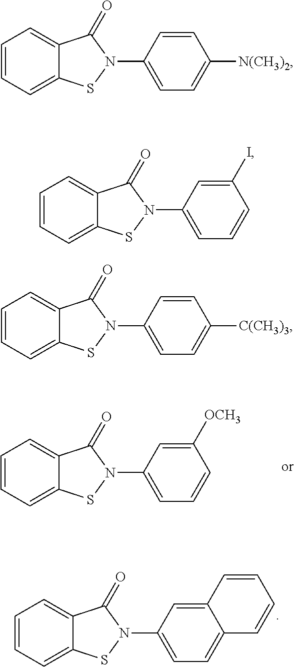

5. The compound of claim 3, wherein the compound is:

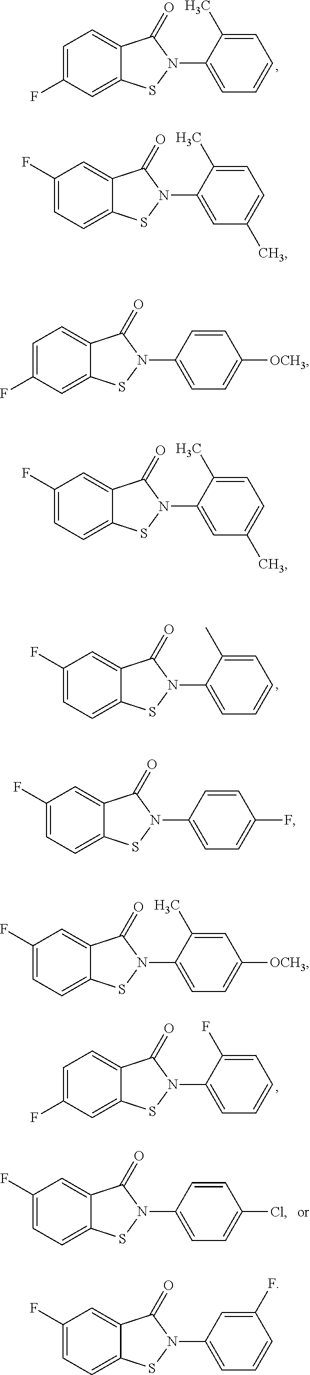

6. A pharmaceutical composition comprising a compound of claim 1 in a pharmaceutically acceptable carrier.

7. A method of modulating the activity of phosphomannomutase 2 (PMM) and phosphomannose isomerase (PMI), the method comprising the step of administering to a subject in need thereof, a therapeutically effective amount of the compound of Formula I of claim 1 or the pharmaceutical composition of claim 6.

8. A method of modulating the activity of phosphomannomutase 2 (PMM), the method comprising the step of administering to a subject in need thereof, a therapeutically effective amount of the compound of Formula I of claim 1 or the pharmaceutical composition of claim 6.

9. A method of modulating the activity of phosphomannose isomerase (PMI), the method comprising the step of administering to a subject in need thereof, a therapeutically effective amount of the compound of Formula I of claim 1 or the pharmaceutical composition of claim 6.

10. A method of inhibiting the activity of phosphomannose isomerase (PMI), the method comprising the step of administering to a subject in need thereof, a therapeutically effective amount of the compound of Formula I of claim 1 or the pharmaceutical composition of claim 6.

11. A method of treating Congenital Disorder of Glycosylation Type Ia (CDG-Ia), the method comprising the step of administering to a subject in need thereof, a therapeutically effective amount of the compound of Formula I of claim 1 or the pharmaceutical composition of claim 6.

12. The method of claim 10, wherein the CDG-Ia is ataxia, seizures, retinopathy, liver fibrosis, coagulapathies, failure to thrive, dysmorphic features, strabismus.

13. The method of claim 10, wherein the CDG-Ia is myopia, infantile esotropia, delayed visual maturation, low vision, optic pallor, and reduced rod function on electrotinography.

14. The method of claim 10, wherein the CDG-Ia is congenital hyperinsulinism with hyperinsulinemic hypoglycemis in infancy.

15. A method of treating an microbial infection, the method comprising the step of administering to a subject in need thereof, a therapeutically effective amount of the compound of Formula I of claim 1 or the pharmaceutical composition of claim 6.

16. The method of claim 15, wherein the microbial infection is a bacterial infection.

17. The method of claim 16, wherein the bacterial infection is a Gram negative bacterial infection.

18. The method of claim 17, wherein the Gram negative bacterial infection is Pseudomonas aeruginosa infection.

19. The method of claim 15, wherein the microbial infection is a fungal infection.

20. The method of claim 19, wherein the fungal infection is a Candida albicans or Cryptococcus neoformans infection.

21. A method for killing bacteria or fungi, wherein the bacteria or fungi are selected from gram-negative bacteria, gram-positive bacteria and yeast, the method comprising the step of administering to a subject in need thereof, a therapeutically effective amount of the compound of Formula I of claim 1 or the pharmaceutical composition of claim 6, wherein the contacting is for a time and under conditions effective to kill bacteria or fungi.

22. The method of claim 21, wherein the bacteria are Gram-negative bacteria.

23. The method of claim 22, wherein the Gram-negative bacteria are selected from Pseudomonas aeruginosa and Escherichia coli.

24. The method of claim 21, wherein the bacteria are Gram-positive bacteria.

25. The method of claim 24, wherein the Gram-positive bacteria are selected from Staphylococcus aureus and Streptococcus faecalis.

26. The method of claim 21, wherein the fungi are Candida albicans or Cryptococcus neoformans.
